(19) United States
(12) Patent Application Publication
(10) Pub. No.: US 2019/0216044 A1
(43) Pub. Date: Jul. 18, 2019

(54) PORTABLE METHOD OF VIDEO RECORDING OF LIFE CYCLES OF INSECTS, SMALL ANIMALS, PLANTS AND CHEMICAL REACTIONS

(71) Applicant: Charles Douglas Mazzapica, San Juan Capistrano, CA (US)

(72) Inventor: Charles Douglas Mazzapica, San Juan Capistrano, CA (US)

(21) Appl. No.: 15/873,841

(22) Filed: Jan. 17, 2018

Related U.S. Application Data
(60) Provisional application No. 62/606,592, filed on Oct. 2, 2017.

(51) Int. Cl. A01K 1/03; H04N 7/18; H04N 5/77; H04N 5/225; A01G 7/00; A01K 29/00; A01G 9/20; A01K 31/14

(52) U.S. Cl. CPC A01K 1/031; H04N 7/183; H04N 5/77; H04N 5/2256; A01G 22/25; A01K 29/005; A01G 9/20; A01K 31/14; A01G 7/00

(57) ABSTRACT

A method of observing the complete lifecycles of living creatures. A portable enclosure is created to match the best environment needed for any life-form to flourish. This enclosure contains lights, video cameras and colorful background scenes to record the lifecycle of the living creature. The video and still photography is then transmitted by WIFI to a smart phone, tablet or computer for review.

A method of creating an adjustable opening dimension for bird enclosures for attracting different species of birds to the enclosure.

A method of observing complete lifecycles of non-living material and living tissue going through changes due to chemical and temperature alterations or living tissue additions.

A portable enclosure is created to match the best environment needed for a material and living tissue observation as the material and living tissue undergoes intentional changes. This enclosure contains lights and video cameras to record the lifecycle of the material or living tissue changes. The video and still photography is then transmitted by WIFI to a smart phone, tablet or computer for review.

### BACKGROUND OF THE INVENTION

The present invention relates generally to the video recording of the lifecycles of small animals, plants and insects, and more particularly, to a method of capturing a complete segment of any lifecycle with normal video, still photography and most important with the use of time-lapse video.

All small animals, plants and insects experience lifecycles as new animals or insects are born or as existing insects or animals go through a metamorphosis from one stage to another. Plant lifecycles start with a seed and grow to maturity. These lifecycles can be captured with photography, real time video and time-lapse video.

All animal and insect lifecycles take place in natural environments. These environments generally take place outside or away from human living establishments. All animals seek private safe environments to reproduce or to stage the metamorphosis segment of their lifecycle.

Numerous species of birds will seek out cavities in any number of natural or man-made structures to reproduce. These lifecycles of birds require a safe environment from weather and predators and normally these lifecycles are out of the view of humans.

Numerous species of insects seek calm quiet dark places to reproduce lay eggs or go through a metamorphosis from infant to adulthood. Humans may catch a glimpse of a single moment of these lifecycles, but not the entire sequence.

All plant life begins as seeds and germinates to a state of roots and above ground foliage. Humans do not see the germination process or the below ground growth of roots. Humans may catch a glimpse of part of the above ground growth, but not the entire sequence.

Humans can watch the complete lifecycles of these species by watching video programs produced by large budget entities who send crews out into natural environments. These crews spend months sometimes years in natural environments to capture these lifecycle with still photography, video and time lapse. This is a procedure that few humans can afford the time to participate in. This is a procedure that few humans can afford the cost to participate in.

Spending time in natural environment for months to capture species lifecycles are not affordable for students of all levels. A large percentage of students neither have the time or the money to participate in such endeavors.

Laboratories observe changes in materials due to temperature or chemical additions or subtractions. Changes in any type of material subjected to many types of artificial environments can be observed. Laboratory personnel set up cameras to observe changes in materials due to temperature changes or chemical additions or subtractions.

Laboratories observe changes to living tissue due to temperature change, chemical additional or subtraction and the addition or subtraction of other living tissue. Changes in any type of living tissue subjected to temperature change or the addition or subtraction of chemical or additional living tissue can be observed. Laboratory personnel set up cameras to observe changes in living tissue due to temperature changes or chemical additions or subtractions.

### BRIEF SUMMARY OF THE INVENTION

For a human to observe the complete lifecycle of many different types of species without spending many months in a natural environment or spending enormous amounts of money, a portable natural environment with video recording devices installed can be used. Thereby, the entire lifecycle of numerous species can be observed and recorded in the home, classroom or laboratory. The invention eliminates the need to spend months in the natural environment to observe and record these lifecycles and the cost of this portable natural environment will be affordable for all.

In one embodiment of the invention, the method of observing and recording a complete life cycle of cavity dwelling birds and any small animal or reptile include the following steps. A small box is designed to attract several different types of birds. The attraction coincided with the bird's natural inclination to procreate.

The bird seeks out a quiet safe place for the procreation to take place. The box is then equipped with one or more video cameras. These video cameras also produce a WIFI signal. Lights are also installed to illuminate the inside of the portable environmental device. Once the bird has made a home in the portable environmental location, then the video recording can begin.

The inside of the box is equipped with the ability to accept an infinite number of colorful scenes of nature. These scenes of nature will act as the background images for the photography, video and time lapse video to be recorded inside the portable enclosure. This is one of the final steps in truly recreating a scene that is usually created in the wilds after spending large sums of money. The scenes will be printed on a series of cards that will be slipped into place inside the box. Every wall will accept a background card scene. Green Screen green will also be used offer the filmmaker the opportunity to use any background with the aid of Chroma key computer editing.

The entire lifecycle of the bird can be observed and recorded. Real time video can be recorded. These video files can be viewed through the WIFI signal from any location. These video files are also being added to the memory card installed in the video camera or cameras. Still photography can also be produced by the same video camera or cameras installed in the portable environmental location. These still photography files can also be transmitted over the WIFI system.

The most important recording will be the time-lapse video which will record the entire lifecycle of the bird occupant of the portable environmental device. The time-lapse video will record for the entire reproduction cycle of the bird or small animal.

This cycle may last for up to three months or longer. After the complete life cycle is recorded over the required time period (two to three months) in the time-lapse format the entire three month cycle can be viewed in five minutes or any short time period designated by the operator of the portable environmental device. After the bird has completed it's reproductive cycle, portable environmental device can be cleaned out and readied for the next occupant.

In one embodiment of the invention, the method of observing and recording a complete life cycle of certain insects include the following steps. A small box is designed to perfectly match the natural environment of any insect that goes through a metamorphosis, for example the lifecycle of a butterfly.

A small box is designed for the perfect environment for the butterfly larva. The box is then equipped with one or more video cameras. These video cameras also produce a WIFI signal. Lights are also installed to illuminate the inside of the portable environmental device. Once the butterfly larva is placed in the portable environmental location or box , then the video recording can begin. The entire lifecycle of the butterfly from larva to butterfly can be observed and recorded.

The inside of the box is equipped with the ability to accept an infinite number of colorful scenes of nature. These scenes of nature will act as the background images for the photography, video and time lapse video to be recorded inside the portable enclosure. This is one of the final steps in truly recreating a scene that is usually created in the wilds after spending large sums of money. The scenes will be printed on a series of cards that will be slipped into place inside the box. Every wall will accept a background card scene. Green Screen green will also be used offer the filmmaker the opportunity to use any background with the aid of Chroma key computer editing.

Real time video can be recorded. These video files can be viewed through the WIFI signal from any location. These video files are also being added to the memory card installed in the video camera. Still photography can also be produced by the same video camera or by a second camera installed in the portable environmental location. These still photography files can also be transmitted over the WIFI system.

The most important recording will be the time-lapse video which will record the entire lifecycle of the insect occupant of the portable environmental device. The time-lapse video will record for the entire reproduction cycle of the insect. This cycle may last for up to three months or longer. After the complete life cycle is recorded over the required time period (two to three months) in the time-lapse format the entire three month cycle can be viewed in five minutes or any short time period designated by the operator of the portable environmental device.

In one embodiment of the invention, the method of observing and recording a complete or partial life cycle of any plant include the following steps. A small box is designed to perfectly match the natural environment that will offer an ideal growing location for the plant species selected to be grown. The box is then equipped with one or more video cameras.

These video cameras also produce a WIFI signal. Lights are also installed to illuminate the inside of the portable environmental device. To observe the underground portion of the plant's lifecycle a piece of glass will be used. Once the plant seed and earth worms are placed in the appropriate soil to start the lifecycle the video and still cameras will be turned on. The entire lifecycle of the plant above and below the ground will be recorded. Real time video can be recorded.

The inside of the box is equipped with the ability to accept an infinite number of colorful scenes of nature. These scenes of nature will act as the background images for the photography, video and time lapse video to be recorded inside the portable enclosure. This is one of the final steps in truly recreating a scene that is usually created in the wilds after spending large sums of money. The scenes will be printed on a series of cards that will be slipped into place inside the box. Every wall will accept a background card scene. Green Screen green will also be used offer the filmmaker the opportunity to use any background with the aid of Chroma key computer editing.

These video files can be viewed through the WIFI signal from any location. These video files are also being added to the memory card installed in the video camera. Still photography can also be produced by the same video cameras installed in the portable environmental location. These still photography files can also be transmitted over the WIFI system.

The most important recording will be the time-lapse video, which will record the entire lifecycle of the plant. The time-lapse video will record for the entire reproduction cycle of the plant. This cycle may last from two weeks to three months or longer. After the complete life cycle is recorded over the required time period (two to three months) in the time-lapse format the entire three month cycle can be viewed in five minutes or any short time period designated by the operator of the portable environmental device.

In one embodiment of the invention, the method of observing and recording a complete or partial life cycle of a beehive include the following steps. A small box is designed to perfectly match the natural environment that will offer an ideal growing environment for a working beehive. The box is then equipped with one or more video cameras.

These video cameras also produce a WIFI signal. Lights are also installed to illuminate the inside of the portable environmental device. To observe the working beehive all of a bee hive's components must bee installed. Once the beehive components and live bees are introduced into the natural environment box video and still cameras will be turned on.

The entire lifecycle of a beehive will be recorded. Real time video can be recorded. These video files can be viewed through the WIFI signal transmitted from the natural environment to any location on the internet. These video files are also being added to the memory card installed in the video camera. Still photography can also be produced by the same video cameras installed in the portable environmental location. These still photography files can also be transmitted over the WIFI system.

The most important recording will be the time-lapse video, which will record any amount of time covering the lifecycle of the bee hive from one week to several years. After the complete life cycle is recorded over the required time period in the time-lapse format the entire three month to several year cycle can be viewed in five minutes or any short time period designated by the operator of the portable environmental device.

In one embodiment of the invention, the method of observing and recording the changes in any type of non-living material for research or education purposes is possible. A box is designed to observe any material or item going through changes created by adding chemicals or altering the temperature or any other type of experiment where change takes place. These changes will be observed and recorded by one or more video cameras. These cameras are recording video, still photos or time lapse video.

In one embodiment of the invention, the method of observing and recording the changes in living tissue of all types for research and education purposes is possible. A box is designed to observe any living tissue undergoing change due to temperature change, chemical addition or adding additional living tissue.

These changes will be observed and recorded by one or more video cameras. These cameras are recording video, still photos or time-lapse video.

### BRIEF DESCRIPTION OF DRAWINGS

These, as well as other features of the present invention, will become apparent upon reference to the drawings wherein:

FIGS. 1 & 2 shows the complete diagram of the natural environmental box for the observation of most birds to complete the lifecycle of new offspring;

FIG. 3 shows the complete diagram of the adjustable opening for bird enclosures. These adjustments offer one bird enclosure the opportunity to attract different species of birds.

FIG. 4 shows the complete diagram of the natural environmental box for the observation a butterfly or other insects to complete the metamorphosis of a new lifecycle;

FIG. 5 shows the complete diagram of the natural environmental box for the observation of all plants to complete their growth cycle from seed germination to fully grown plant.

### DETAILED DESCRIPTION OF THE INVENTION

As mentioned above, most life forms have a process of creating new offspring and this process involves several steps by the existing parents of the new life form or by certain procedures performed by humans. Once all the necessary procedures have been set into motion the new life form goes through the several steps to achieve adulthood or mature status. The portable natural environment equipped with video cameras can record all these steps for instant observations or review at a later date.

To achieve the ability to record the natural process of any species, special procedures must be added to the portable natural environment so each life form can first of all survive infancy and then grow to maturity or adulthood. Each life form that will inhabit the portable natural environment will require its own special treatment and additions so survival and growth to maturity is assured. Each life form that has been identified to grow in the portable natural environment will now each be described in detail.

Once the portable natural environment has been readied for the living occupant, the box itself must be made ready for the best quality video recordings, audio, still photography and time-lapse video.

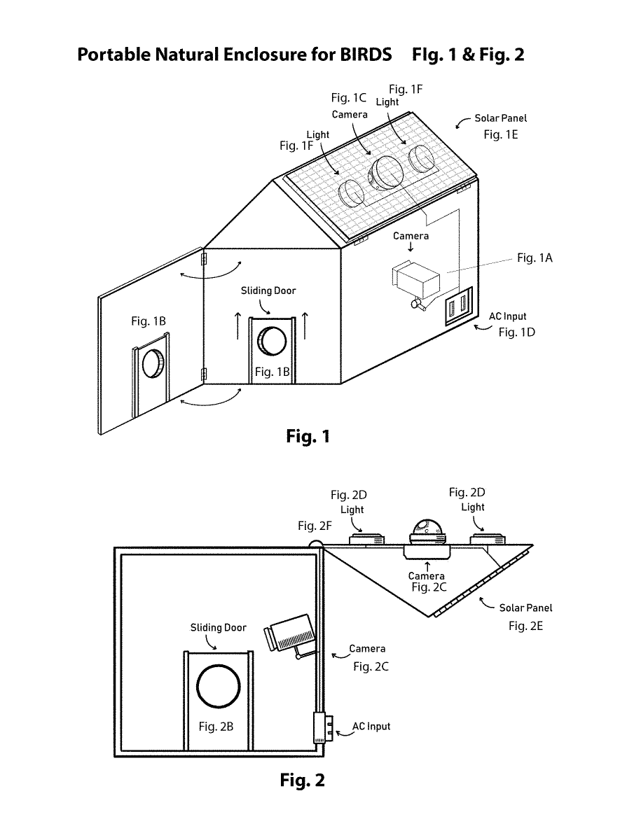

FIG. 1 shows the portable natural environment designed for birds. This environment requires a structure that will survive being placed in the outdoors. The building material can be of any type that will form a box big enough to house a bird family during their time of procreation. These construction materials may consist of wood, plastic, cardboard, tin or any material.

The design of this portable natural environment is similar to any bird house with a size consisting of 8×8×8 inches to 16×12×12 inches. (FIG. 1) The walls and top of these portable natural environments open up to provide access to the components inside. (FIG. 2) A complete side-wall or a portion of the wall is made to swing open to reveal the inner working of the environment. (FIG. 1B & 2B)

The design also enables the top of the environment to swing open. (FIG. 2F) The top of the bird house may contain components of the operation of the natural Environment and opening of the top will offer access to these components.

The inside of the box is equipped with the ability to accept an infinite number of colorful scenes of nature. These scenes of nature will act as the background images for the photography, video and time lapse video to be recorded inside the portable enclosure. This is one of the final steps in truly recreating a scene that is usually created in the wilds after spending large sums of money. The scenes will be printed on a series of cards that will be slipped into place inside the box. Every wall will accept a background card scene. Green Screen green will also be used offer the filmmaker the opportunity to use any background with the aid of Chroma key computer editing.

All birdhouses include an opening for the bird family to enter and exit the enclosure. (FIG. 2B) All cavity dwelling birds seeks out the perfect size opening to the cavity that will be used to procreate. The size of the opening is determined to assure as much safety as possible for the bird family against predators.

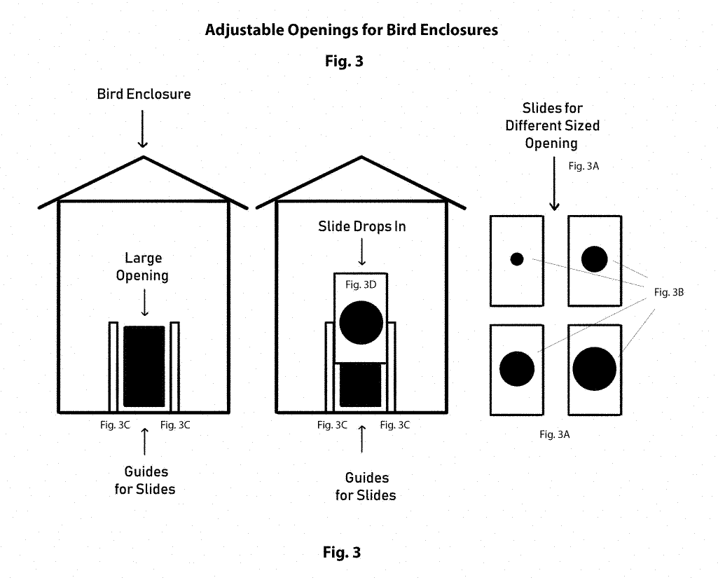

The portable natural environment (FIG. 3) has a unique system to alter the size of the opening. The operator can determine the size of the opening. The operator can determine what type of bird they would like to occupy their portable natural environment or determine which bird species are available in their geographical region.

This selection can be determined by selecting one of several “opening slides” included with the Portable Natural Enclosure (FIG. 3A). Each slide contains an opening and each slide's opening is a different size circle (FIG. 3B). These sizes range from ½ inch to 4 inches. The opening slide (FIG. 3D) is inserted into the runners or guides (FIG. 3C) on the inside or outside of the Portable Natural Environment enclosure thus creating the exact size opening required. These slides can be switched out after each bird family has used the enclosure to attract a different type of bird for the next bird encounter.

The Portable Natural Enclosure can resemble a traditional birdhouse on the outside, but the inside is where portable natural environment is completely different from any other bird enclosure. The number one addition is high quality video cameras (FIG. 1A, FIG. 1C, FIG. 2C) that produce real time video, still photography and time lapse video. Also inside are the colorful backgrounds attached to each wall.

The number of cameras can range from one to six. The cameras are positioned to capture the best possible angle for the best possible observation of the bird and bird family. The camera will be able to record video in time-lapse mode for one week to four months.

The basic camera set-up (FIG. 1D) inside the enclosure will involve at least two locations. “Camera One” will be located 6 inches from the bottom and centered on wall #2 (FIG. 1G). The lens will be pointed at the center of the enclosure. This view will capture all activity of the birds which will include; entering the enclosure with any nest building material, building a nest, laying eggs, incubating the eggs, hatching the eggs, feeding the new offspring, teaching new offspring the ways of being a bird and finally leaving the nest for parts unknown.

“Camera two” will be located in the roof of the enclosure and will be pointed straight down for an aerial view of the bird activity. (FIG. 2C)

All video and still photography files recorded from inside the enclosure will be transmitted via WIFI or Bluetooth technology to the portable natural environment owner's smart phone, tablet or computer. All files will also be recorded to a memory card contained in the camera. These files can be downloaded at a later date.

The interior of the portable natural enclosure will have one to four sets of lights placed strategically to produce the best possible video and still photography of the bird occupants performing their new lifecycle. (FIG. 1F & FIG. 2D) The lights will mirror the natural light cycle found outside of the bird house as far as a schedule of off and on.

The interior of the portable natural enclosure will also contain a clock. This clock will show day of week, date of moth, hour, minutes and am or pm. As the time lapse camera records video over weeks or months the clock will register real time values in playback mode.

Lights and cameras will be powered by USB power. Interior walls of The Portable Natural Enclosures will work as conduits for the power lines that feed the cameras and lights the electricity to function. A USB power junction will be located in the back corner (FIG. h) where a AC power outlet will be fitted through the walls for AC power to be supplied form the outside of the Portable Natural Enclosure. (FIG. 1D)

Power can also be supplied by solar panels attached to the roof of the enclosure. The solar panel would supply power to all cameras and lights. (FIG. 1E & FIG. 2E)

Audio will also be recorded to capture any sounds created by the life form. These audio files will be transmitted by WiFi along with the video files.

Each interior wall and the floor of the bird enclosure will contain colorful images to create a beautiful setting for the video and still photography.

FIG. 3 shows the portable natural environment designed for insects that go through a Metamorphosis phase in their life cycle. All species of butterflies and other insects (Honey Bees, Termites, Ants and so on) go through such a transformation. The Insect Portable Natural Enclosure is designed for these types of insects. This type of enclosure can also be adjusted to capture video of the changes in living tissue and non-living material.

This environment requires a structure that can be made from any type of material, but will not have to withstand outside forces as this enclosure will be used inside schools, research labs or homes.

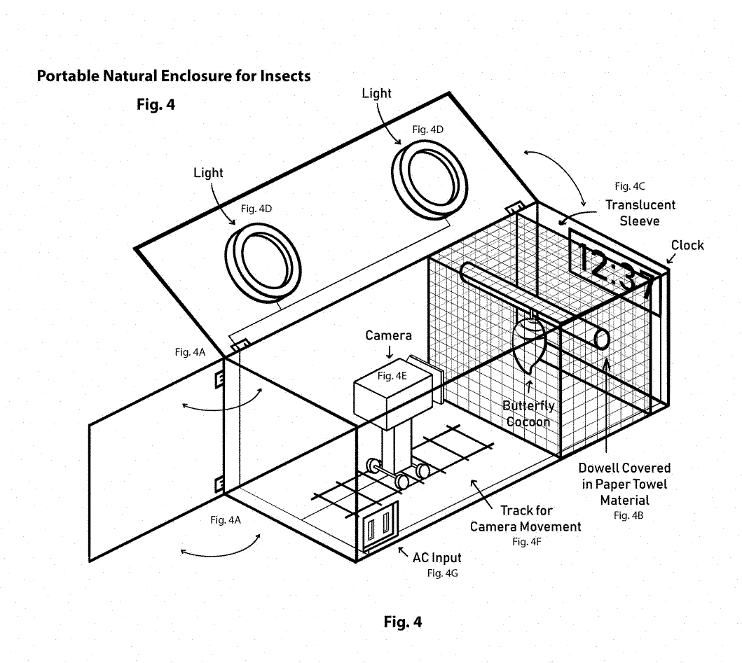

The ideal construction material is waxed coated cardboard, ⅛th to ¼ inch thick. This material creates a structure measuring 8×8×8 inches or other dimensions. The construction will be one or several attached pieces of cardboard that will fold out from a flat beginnings to a three dimensional space of 8×8×8 or other dimensions. The assembled enclosure will have swing open doors from the side and the top. These doors will provide access to the cameras and lights of the portable natural enclosure. (FIG. 4A)

The inside of the box is equipped with the ability to accept an infinite number of colorful scenes of nature. These scenes of nature will act as the background images for the photography, video and time lapse video to be recorded inside the portable enclosure. This is one of the final steps in truly recreating a scene that is usually created in the wilds after spending large sums of money. The scenes will be printed on a series of cards that will be slipped into place inside the box. Every wall will accept a background card scene. Green Screen green cards will also be used offer the filmmaker the opportunity to use any background with the aid of Chroma key computer editing.

Inside the enclosure one small dowel is suspended across the interior. (FIG. 4B) This dowel is to be ⅛th inches thick and span the inside from one wall to the other. This dowel is the perch that the caterpillars attach themselves to for the metamorphosis live cycle. The dowel is perfectly situated to provide the best video and still photography of the metamorphosis process. The dowel is wrapped in a paper towel type fabric. This allows to for easier attachment by the caterpillar. The dowel may also be a replica of a real tree branch. This type of hanger for the caterpillars combined with the colorful real natural backgrounds will produce amazing natural looking video and photography.

A sleeve with dimensions of one inch thick by eight inches tall and eight inches wide is made up of clear plastic material is fitted over the dowel. (FIG. 4C) This sleeve runs from the bottom of the enclosure to just above the dowel. This sleeve will provide a guide to the caterpillars to the dowel. Once all caterpillars have attached themselves to the dowel, the sleeve can be removed. This will offer a clear view of the living insects for the enclosed video cameras.

Lights (FIG. 4D) are positioned inside of the portable natural enclosure to provide the best possible video of the insect lifecycle.

The video camera (FIG. 4E) is position half way up from the bottom attached to a wall and pointed straight into the middle of the enclosure. Straight at the dowels where the caterpillars will start their journey of metamorphosis.

The video camera is attached to a device that allows the camera to move on a track. (FIG. 4F) The camera would be able to be moved in towards the living creatures while they go through the metamorphosis process. Moving the camera in would allow for extreme close-ups.

Time- lapse video will capture the entire lifecycle process of the insects. Once the complete lifecycle is compete the butterfly simply flies away to the next phase of their lives. The only item that remains is the video of the entire process.

This entire process can be set up for an ant colony, bee colony, termite colony and other insects also.

All video and still photography files recorded from inside the enclosure will be transmitted via WIFI or Bluetooth technology to the portable natural environment owner's smart phone, tablet or computer. All video files will also be recorded to a memory card contained in the camera or cameras. These files can be downloaded at a later date.

Audio will also be recorded to capture any sounds created by the life form. These audio files will be transmitted along with the video files.

Lights and cameras will be powered by USB power. Interior walls of The Portable Natural Enclosures will work as conduits for the power lines that feed the cameras and lights the electricity to function. A USB power junction will be located in the back corner (FIG. 4G) where a AC power outlet will be fitted through the walls for AC power to be supplied from the outside of the Portable Natural Enclosure.

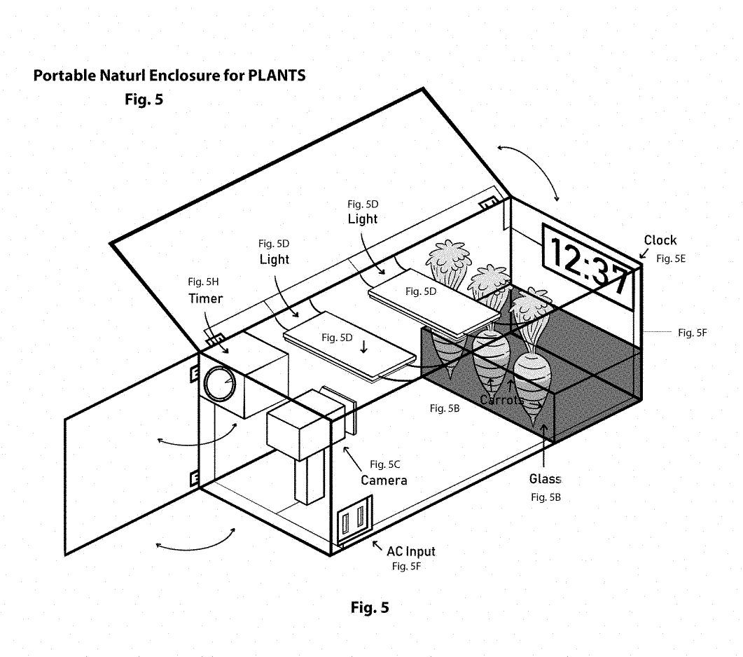

FIG. 5 shows the portable natural environment designed for plants that demonstrate a lifecycle from seeds to a mature plant. The Agricultural Portable Natural Enclosure is designed for these types of plants.

This environment requires a structure that can be made from any type of material, but will not have to withstand outside forces as this enclosure will be used inside schools, research labs or homes.

The ideal construction material is waxed coated cardboard, ⅛th to ¼″ inch thick. This material creates a structure measuring 16×12×12 inches. The construction will be a one or several attached pieces that will fold out from flat to a three dimensional space of 16×12×12 from a flat pre-construction state. The enclosure will have swing open doors (FIG. 5A) from one of the vertical walls and from the top of the enclosure. These doors will provide access to the seeds and plants and the cameras and lights of the portable natural enclosure.

Inside the enclosure a piece of glass or any translucent material measuring six by twelve inches (FIG. 5B) is installed on the floor of the enclosure four inches from the back wall (FIG. 5F).

This piece of glass will allow the visual inspection of the plant growth below the surface.
The video cameras will be able to view the plant's lifecycle above and below ground level.

Soil is placed in the space in-between the glass and the back wall (FIG. 5F). Seeds are planted inch below the surface of the soil and placed against the glass surface. The seed is now visible behind the glass.

The camera (FIG. 5C) is placed against the front wall (FIG. 5A) and is pointed at point directly down the center of the enclosure. This position looks directly into the center of the piece of glass. The view is above and below the surface of the soil. The camera offers the perfect situated to provide the best video and still photography of the plant's lifecycle from seed and mature plant.

Lights (FIG. 5D) are positioned inside of the portable natural enclosure to provide the best possible video of the plant lifecycle.

Time-lapse video will capture the entire lifecycle process of the plant. Once the complete lifecycle is compete and outgrows the enclosure the plant can be removed. The only item that remains is the video of the entire process.

The interior of the portable natural enclosure will also contain a clock. This clock (5E) will show day of week, date of moth, hour, minutes and am or pm. As the time lapse camera records video over weeks or months the clock will register real time values in playback mode. A timer (FIG. 5H) will also control the lights so they will mimic the natural cycle of light and dark in the natural world.

All video and still photography files recorded from inside the enclosure will be transmitted via WIFI or Bluetooth technology to the portable natural environment owner's smart phone, tablet or computer. All files will also be recorded to a memory card contained in the camera. These files can be downloaded at a later date.

Lights and cameras will be powered by USB power. Interior walls of The Portable Natural Enclosures will work as conduits for the power lines that feed the cameras and lights the electricity to function. A USB power junction will be located in the back corner (FIG. 5F) where a AC power outlet will be fitted through the walls for AC power to be supplied from the outside of the Portable Natural Enclosure.

What is claimed is:

1. A method of observing the complete lifecycle of insects, small animals, plants and to observe changes in living tissue and non-living materials, comprising:
Creating a portable enclosure to house the insect, animal, plant, living tissue or non-living materials; provide the best environment for insects, small animals, plants, living tissue and non-living materials; provide a video camera and lights and colorful real natural backgrounds in the environment of small animals, insects, plants and materials; providing
the ability to record video, still photos and time lapse video of the lifecycles of small animals, insects, plants, living tissue and non-living materials changes during chemical or temperatures changes.

2. The method according to claim 1, wherein the insects, small animals, plants and materials will all go through changes.

3. The method according to claim 2, wherein the changes are to be observed.

4. The method according to claim 3, wherein the environment for each the insect, small animals, plants and materials is setup in a manner for each to flourish with the best possible results.

5. The method according to claim 4, wherein each environment is set-up with appropriate lighting to produce the best possible still photography and video.

6. The method according to claim 5, wherein each environment contains colorful images on the inside walls to match real natural locations.

7. The method according to claim 6, wherein each environment contains one or more cameras to record the observations with the best possible still photography, video and time-lapse video.

8. The method according to claim 6, wherein all video and still photography is transmitted by WIFI to a smartphone, tablet or computer.

9. The method of altering the opening of any size bird enclosure (bird house) and thus offering the ability to attract many different species of birds.

10. The method according to claim 8, wherein the entry point of any bird enclosure can be altered by using slides with pre-cut circular dimensions.

11. The method according to claim 9, wherein the bird enclosure is constructed with slots to accept rectangular slides. Each slide contains a pre-cut circular dimension.

12. The method according to claim 10, wherein the appropriate slide for the appropriate bird species is inserted into the slots of the bird enclosure to create an adjustable bird entry point.
